(19) United States
(12) Patent Application Publication
(10) Pub. No.: US 2011/0033390 A1
(43) Pub. Date: Feb. 10, 2011

(54) MR IMAGING AGENT OR MEDIUM COMPRESSING HYPERPOLARISED 13C ALANINE AND METHODS OF IMAGING WHEREIN SUCH AN IMAGING MEDIUM IS USED

(76) Inventors: Anna Gisselsson, Lund (SE); Georg Hansson, Velinge (SE); Sven Mansson, Bjarred (SE); Rene In't Zandt, Sodra Sandby (SE); Magnus Karlsson, Malmo (SE); Pernille R. Jensen, Koberhavn (DK); Mathilde H. Lerche, Fredriksberg (DK)

Correspondence Address: GE HEALTHCARE, INC., IP DEPARTMENT 101 CARNEGIE CENTER, PRINCETON, NJ, 08540-6231, US

(21) Appl. No.: 12/864,537

(22) PCT Filed: Feb. 3, 2009
(86) PCT No.: PCT/EP09/51174

§ 371 Date: Sep. 28, 2010

(30) Foreign Application Priority Data
Feb. 4, 2008 (EP) 08002002.7

(51) Int. Cl. A61K 49/20; A61K 49/06; C12Q 1/02; C07C 229/08

(52) U.S. Cl. 424/9.36; 424/9.3; 435/29; 562/576

(57) ABSTRACT

The invention relates to hyperpolarised ¹³C-alanine, its use as imaging agent, an imaging medium comprising hyperpolarised ¹³C-alanine and methods of ¹³C-MR detection wherein such an imaging medium is used. Further, the invention relates to methods of producing hyperpolarised ¹³C-alanine.

The invention relates to hyperpolarised ¹³C-alanine, its use as imaging agent, an imaging medium comprising hyperpolarised ¹³C-alanine and methods of ¹³C-MR detection wherein such an imaging medium is used. Further, the invention relates to methods of producing hyperpolarised ¹³C-alanine.

Magnetic resonance (MR) imaging (MRI) is a technique that has become particularly attractive to physicians as images of a patients body or parts thereof can be obtained in a non-invasive way and without exposing the patient and the medical personnel to potentially harmful radiation such as X-rays. Because of its high quality images and good spatial and temporal resolution, MRI is a favourable imaging technique for imaging soft tissue and organs.

MRI may be carried out with or without MR contrast agents. However, contrast-enhanced MRI usually enables the detection of much smaller tissue changes which makes it a powerful tool for the detection of early stage tissue changes like for instance small tumours or metastases.

Several types of contrast agents have been used in MRI. Water-soluble paramagnetic metal chelates, for instance gadolinium chelates like Omniscan™ (GE Healthcare) are widely used MR contrast agents. Because of their low molecular weight they rapidly distribute into the extracellular space (i.e. the blood and the interstitium) when administered into the vasculature. They are also cleared relatively rapidly from the body.

Blood pool MR contrast agents on the other hand, for instance superparamagnetic iron oxide particles, are retained within the vasculature for a prolonged time. They have proven to be extremely useful to enhance contrast in the liver but also to detect capillary permeability abnormalities, e.g. “leaky” capillary walls in tumours which are a result of tumour angiogenesis.

Despite the undisputed excellent properties of the aforementioned contrast agents their use is not without any risks. Although paramagnetic metal chelates have usually high stability constants, it is possible that toxic metal ions are released in the body after administration. Further, these type of contrast agents show poor specificity.

WO-A-99/35508 discloses a method of MR investigation of a patient using a hyperpolarised solution of a high T₁ agent as MRI contrast agent. The term “hyperpolarisation” means enhancing the nuclear polarisation of NMR active nuclei present in the high T₁ agent, i.e. nuclei with non-zero nuclear spin, preferably ¹³C- or ¹⁵N-nuclei. Upon enhancing the nuclear polarisation of NMR active nuclei, the population difference between excited and ground nuclear spin states of these nuclei is significantly increased and thereby the MR signal intensity is amplified by a factor of hundred and more. When using a hyperpolarised ¹³C- and/or ¹⁵N-enriched high T₁ agent, there will be essentially no interference from background signals as the natural abundance of ¹³C and/or ¹⁵N is negligible and thus the image contrast will be advantageously high. The main difference between conventional MRI contrast agents and these hyperpolarised high T₁ agents is that in the former changes in contrast are caused by affecting the relaxation times of water protons in the body whereas the latter class of agents can be regarded as non-radioactive tracers, as the signal obtained arises solely from the agent.

A variety of possible high T₁ agents for use as MR imaging agents are disclosed in WO-A-99/35508, including non-endogenous and endogenous compounds. As examples of the latter intermediates in normal metabolic cycles are mentioned which are said to be preferred for imaging metabolic activity. By in vivo imaging of metabolic activity, information of the metabolic status of a tissue may be obtained and said information may for instance be used to discriminate between healthy and diseased tissue.

For instance pyruvate is a compound that plays a role in the citric acid cycle and the conversion of hyperpolarised ¹³C-pyruvate to its metabolites hyperpolarised ¹³C-lactate, hyperpolarised ¹³C-bicarbonate and hyperpolarised ¹³C-alanine can be used for in vivo MR studying of metabolic processes in the human body.

The metabolic conversion of hyperpolarised ¹³C-pyruvate to its metabolites hyperpolarised ¹³C-lactate, hyperpolarised ¹³C-bicarbonate and hyperpolarised ¹³C-alanine can be used for in vivo MR study of metabolic processes in the human body since said conversion has been found to be fast enough to allow signal detection from the parent compound, i.e. hyperpolarised ¹³C₁-pyruvate, and its metabolites. The amount of alanine, bicarbonate and lactate is dependent on the metabolic status of the tissue under investigation. The MR signal intensity of hyperpolarised ¹³C-lactate, hyperpolarised ¹³C-bicarbonate and hyperpolarised ¹³C-alanine is related to the amount of these compounds and the degree of polarisation left at the time of detection, hence by monitoring the conversion of hyperpolarised ¹³C-pyruvate to hyperpolarised ¹³C-lactate, hyperpolarised ¹³C-bicarbonate and hyperpolarised ¹³C-alanine it is possible to study metabolic processes in vivo in the human or non-human animal body by using non-invasive MR imaging and/or MR spectroscopy.

The MR signal amplitudes arising from the different pyruvate metabolites vary depending on the tissue type. The unique metabolic peak pattern formed by alanine, lactate, bicarbonate and pyruvate can be used as fingerprint for the metabolic state of the tissue under examination.

Hyperpolarised ¹³C-pyruvate may for instance be used as an MR imaging agent for assessing the viability of myocardial tissue by MR imaging as described in detail in WO-A-2006/054903 and for in vivo tumour imaging as described in detail in WO-A-2006/011810.

Tumour tissue is often characterised by an increased perfusion and higher metabolic activity. The process of increasing the vascular bed, angiogenesis, is induced by cells that due to their higher metabolic needs and/or their larger distance from a capillary are not able to get enough substrates that can provide the energy needed to sustain energy homeostasis. It is in this area, where cells have problems in producing enough energy a marked change in metabolic pattern is expected. Tissue with problems sustaining energy homeostasis will alter its energy metabolism which in particular results in an increased lactate production. With the use of hyperpolarised ¹³C-pyruvate as an MR imaging agent, this higher metabolic activity can be seen by an increased production of ¹³C-lactate which can be detected by ¹³C-MR detection. However, since the production of hyperpolarised ¹³C-pyruvate which is suitable as an in vivo imaging agent is not without challenges, there is a need of alternative hyperpolarised imaging agents which can be used to obtain information about metabolic activity.

We have now found that hyperpolarised ¹³C-alanine may be used as such an imaging agent.

Alanine reacts with α-ketoglutarate to form pyruvate and glutamate, the reaction is catalysed by alanine transaminase. Further, pyruvate is formed by the reaction of alanine with glyoxylate. This reaction is catalysed by alanine-glyoxylate transaminase. Both enzymes exist in cytosolic and mitochondrial isoforms. Hence by using hyperpolarised ¹³C-alanine as an imaging agent, the metabolic activity can be assessed.

Thus in a first aspect the invention provides an imaging medium comprising hyperpolarised ¹³C-alanine

The term “imaging medium” denotes a liquid composition comprising hyperpolarised ¹³C-alanine as the MR active agent, i.e. imaging agent.

The imaging medium according to the invention may be used as an imaging medium for in vivo ¹³C-MR detection, i.e. in living human or non-human animal beings. Further, the imaging medium may be used as imaging medium for in vitro ¹³C-MR detection, e.g. of cell cultures, samples like for instance urine, saliva or blood, ex vivo tissue, for instance ex vivo tissue obtained from a biopsy or isolated organs. In a preferred embodiment, the imaging medium according to the invention may be used as an imaging medium for in vivo ¹³C-MR detection

The term “¹³C-MR detection” denotes ¹³C-MR imaging or ¹³C-MR spectroscopy or combined ¹³C-MR imaging and ¹³C-MR spectroscopy, i.e. ¹³C-MR spectroscopic imaging. The term further denotes ¹³C-MR spectroscopic imaging at various time points.

The term “¹³C-alanine” denotes 2-amino-propanoic acid which is isotopically enriched with ¹³C.

The isotopic enrichment of the hyperpolarised ¹³C-alanine is preferably at least 75%, more preferably at least 80% and especially preferably at least 90%, an isotopic enrichment of over 90% being most preferred. Ideally, the enrichment is 100%. Generally, hyperpolarised ¹³C-alanine according to the invention may be isotopically enriched at any carbon atom in the molecule. However, to achieve a long T1, it is preferred that ¹³C-alanine is isotopically enriched with ¹³C at the C1-position (in the following denoted ¹³C₁-alanine) or at the C2-position (in the following denoted ¹³C₂-alanine) Multiple enrichment is also possible like isotopic enrichment at both the C1- and C2-position (in the following denoted ¹³C_{1,2}-alanine), at the C1- and the C3-position (in the following denoted ¹³C_{1,3}-alanine), at the C2- and the C3-position (in the following denoted ¹³C_{2,3}-alanine) or at the C1-, C2- and C3-position (in the following denoted ¹³C_{1,2,3}-alanine) Isotopic enrichment at the C1-position is preferred.

The terms “hyperpolarised” and “polarised” are used interchangeably hereinafter and denote a nuclear polarisation level in excess of 0.1%, more preferred in excess of 1% and most preferred in excess of 10%.

The level of polarisation may for instance be determined by solid state ¹³C-NMR measurements in solid hyperpolarised ¹³C-alanine, e.g. solid hyperpolarised ¹³C-alanine obtained by dynamic nuclear polarisation (DNP) of ¹³C-alanine. The solid state ¹³C-NMR measurement preferably consists of a simple pulse-acquire NMR sequence using a low flip angle. The signal intensity of the hyperpolarised ¹³C-alanine in the NMR spectrum is compared with signal intensity of ¹³C-alanine in a NMR spectrum acquired before the polarisation process. The level of polarisation is then calculated from the ratio of the signal intensities of before and after polarisation.

In a similar way, the level of polarisation for hyperpolarised ¹³C-alanine in solution may be determined by liquid state NMR measurements. Again the signal intensity of the hyperpolarised ¹³C-alanine in solution is compared with the signal intensity of the ¹³C-alanine in solution before polarisation. The level of polarisation is then calculated from the ratio of the signal intensities of ¹³C-alanine before and after polarisation.

Hyperpolarisation of NMR active ¹³C-nuclei may be achieved by different methods which are for instance described in described in WO-A-98/30918, WO-A-99/24080 and WO-A-99/35508, and which all are incorporated herein by reference and hyperpolarisation methods known in the art are polarisation transfer from a noble gas, “brute force”, spin refrigeration, the parahydrogen method and dynamic nuclear polarisation (DNP).

Hyperpolarised ¹³C-alanine can be obtained by directly polarising ¹³C-alanine or by polarising a salt of ¹³C-alanine and subsequent conversion (neutralization) of the salt to ¹³C-alanine with a base or acid. Suitable salts of ¹³C-alanine are commercially available or can be prepared from commercially available ¹³C-alanine and will be discussed in detail in the following paragraphs.

One way for obtaining hyperpolarised ¹³C-alanine is the polarisation transfer from a hyperpolarised noble gas which is described in WO-A-98/30918. Noble gases having non-zero nuclear spin can be hyperpolarised by the use of circularly polarised light. A hyperpolarised noble gas, preferably He or Xe, or a mixture of such gases, may be used to effect hyperpolarisation of ¹³C-nuclei. The hyperpolarised gas may be in the gas phase, it may be dissolved in a liquid/solvent, or the hyperpolarised gas itself may serve as a solvent. Alternatively, the gas may be condensed onto a cooled solid surface and used in this form, or allowed to sublime. Intimate mixing of the hyperpolarised gas with ¹³C-alanine is preferred.

Another way for obtaining hyperpolarised ¹³C-alanine is that polarisation is imparted to the ¹³C-nuclei of alanine by thermodynamic equilibration at a very low temperature and high field. Hyperpolarisation compared to the operating field and temperature of the NMR spectrometer is effected by use of a very high field and very low temperature (brute force). The magnetic field strength used should be as high as possible, suitably higher than 1 T, preferably higher than 5 T, more preferably 15 T or more and especially preferably 20 T or more. The temperature should be very low, e.g. 4.2 K or less, preferably 1.5 K or less, more preferably 1.0 K or less, especially preferably 100 mK or less.

Another way for obtaining hyperpolarised ¹³C-alanine is the spin refrigeration method. This method covers spin polarisation of a solid compound or system by spin refrigeration polarisation. The system is doped with or intimately mixed with suitable crystalline paramagnetic materials such as Ni²⁺, lanthanide or actinide ions with a symmetry axis of order three or more. The instrumentation is simpler than required for DNP with no need for a uniform magnetic field since no resonance excitation field is applied. The process is carried out by physically rotating the sample around an axis perpendicular to the direction of the magnetic field. The pre-requisite for this method is that the paramagnetic species has a highly anisotropic g-factor. As a result of the sample rotation, the electron paramagnetic resonance will be brought in contact with the nuclear spins, leading to a decrease in the nuclear spin temperature. Sample rotation is carried out until the nuclear spin polarisation has reached a new equilibrium.

In a preferred embodiment, DNP (dynamic nuclear polarisation) is used to obtain hyperpolarised ¹³C-alanine. In DNP, polarisation of MR active nuclei in a compound to be polarised is affected by a polarisation agent or so-called DNP agent, a compound comprising unpaired electrons. During the DNP process, energy, normally in the form of microwave radiation, is provided, which will initially excite the DNP agent. Upon decay to the ground state, there is a transfer of polarisation from the unpaired electron of the DNP agent to the NMR active nuclei of the compound to be polarised, e.g. to the ¹³C nuclei in ¹³C-alanine. Generally, a moderate or high magnetic field and a very low temperature are used in the DNP process, e.g. by carrying out the DNP process in liquid helium and a magnetic field of about 1 T or above. Alternatively, a moderate magnetic field and any temperature at which sufficient polarisation enhancement is achieved may be employed. The DNP technique is for example further described in WO-A-98/58272 and in WO-A-01/96895, both of which are included by reference herein.

To polarise a chemical entity, i.e. compound, by the DNP method, a composition of the compound to be polarised and a DNP agent is prepared which is then optionally frozen and inserted into a DNP polariser (where it will freeze if it has not been frozen before) for polarisation. After the polarisation, the frozen solid hyperpolarised composition is rapidly transferred into the liquid state either by melting it or by dissolving it in a suitable dissolution medium. Dissolution is preferred and the dissolution process of a frozen hyperpolarised composition and suitable devices therefore are described in detail in WO-A-02/37132. The melting process and suitable devices for the melting are for instance described in WO-A-02/36005.

In order to obtain a high polarisation level in the compound to be polarised said compound and the DNP agent need to be in intimate contact during the DNP process. This is not the case if the composition crystallizes upon being frozen or cooled. To avoid crystallization, either glass formers need to be present in the composition or compounds need to be chosen for polarisation which do not crystallize upon being frozen but rather form a glass.

The term “glass former” in the context of this application means a chemical compound that, when added to a solution, e.g. a solution according to step a) of the method of the invention, promotes vitrification and prevents crystallization of said solution when it is cooled or frozen. Examples of preferred glass formers in the context of the invention are glycols, i.e. alcohols containing at least two hydroxyl groups, such as ethylene glycol, propylene glycol and glycerol or DMSO.

In one embodiment, ¹³C-alanine, preferably ¹³C₁-alanine is used as a starting material to obtain hyperpolarised ¹³C-alanine by the DNP method. In another preferred embodiment, a salt of ¹³C-alanine, preferably ¹³C₁-alanine is used as a starting material to obtain hyperpolarised ¹³C-alanine by the DNP method.

In a first embodiment, ¹³C-alanine, preferably ¹³C₁-alanine is used as a starting material to obtain hyperpolarised ¹³C-alanine by the DNP method. ¹³C-alanine is a commercially available compound. In a second embodiment, a salt of ¹³C-alanine, preferably a salt of ¹³C₁-alanine is used as a starting material to obtain hyperpolarised ¹³C-alanine by the DNP method. Suitable salts of ¹³C-alanine are for instance ammonium salts of ¹³C-alanine or carboxylate salts of ¹³C-alanine. An ammonium salt of ¹³C-alanine is a chemical entity which comprises as a cation ¹³C-alaninium, for instance ¹³C₁-alaninium i.e. H₃N⁺—C(CH₃)(H)—¹³COOH. A carboxylate salt of ¹³C-alanine is a chemical entity which comprises as an anion 2-aminopropanoate, for instance ¹³C₁-2-aminopropanoate, i.e. H₂N—C(CH₃)(H)—¹³COO⁻.

Ammonium salts of ¹³C-alanine are either commercially available compounds or can generally be obtained by reacting ¹³C-alanine with an acid. In principal any acid that has a lower pKa than the carboxyl group ¹³C-alanine can be used to convert it into its ammonium salt. Solubility of the ammonium salt may be hampered if the counter ion of the acid used to obtain the ammonium salt is either large or lipophilic. More preferred acids are strong acids, even more preferred strong mineral acids like hydrochloric acid (HCl), hydrobromic acid (HBr), hydroiodic acid (HI) or sulphuric acid (H₂SO₄). The most preferred acid is HCl since it is cheap and readily available. By reacting ¹³C-alanine with HCl, an ammonium chloride, i.e. alaninium chloride is obtained. If the hyperpolarised ¹³C-alanine is used for in vivo MR alaninium chloride is a preferred starting material since chloride ions are well tolerated by the human or non-human animal body. However, if for any reason a less well tolerated anion is used, said anion may be exchanged after polarisation by a physiologically very well tolerated anion like chloride by methods known in the art like the use of an anion exchange column. One such reason could be that a ¹³C-alanine sample with higher concentration and/or higher polarisation levels in ¹³C-alanine can be obtained by using a specific acid for the preparation of the ammonium salt of ¹³C-alanine. As an example by using HI a highly concentrated ¹³C-alanine sample can be obtained but iodide is not a preferred anion when it comes to physiological tolerability. Hence said iodide may be exchanged with an anion which is better tolerated, e.g. chloride.

In the method of the invention, if the ammonium salt of ¹³C-alanine is not a commercially available compound, it may either be prepared and isolated or prepared in situ without isolating the obtained ammonium salt. The advantage of isolating the ammonium salt is that the isolated salt can be characterized and it can be determined how much of the ¹³C-alanine was actually converted into the ammonium salt. Further, if other solvents are used in the DNP process than for the preparation of the ammonium salt, it is preferred to isolate the ammonium salt as well.

Carboxylate salts of ¹³C-alanine can generally be obtained by reacting ¹³C-alanine with a base. In principal any base that is a stronger base than the amino group in ¹³C-alanine can be used to convert it into its respective carboxylate salt. Again solubility of the carboxylate may be hampered if the counter ion of the acid used to obtain the carboxylate salt is either large or lipophilic. Preferred bases are inorganic bases, more preferred aqueous solutions of alkali metal or earth alkali metal hydroxides, like aqueous solutions of NaOH, KOH, CsOH, Ca(OH)₂ or Sr(OH)₂. The most preferred base is NaOH since it is cheap and readily available. By reacting ¹³C-alanine with NaOH, a sodium carboxylate, i.e. sodium ¹³C-2-aminopropanoate, is obtained. If the hyperpolarised ¹³C-alanine is used for in vivo MR, sodium ¹³C-2-aminopropanoate is a preferred starting material since sodium cations are well tolerated by the human or non-human animal body. However, if for any reason a less well tolerated cation is used, said cation may be exchanged after hyperpolarisation by a physiologically very well tolerated cation like Na⁺ or meglumine cation by methods known in the art like the use of a cation exchange column. One such reason could be that higher concentrated ¹³C-alanine sample and/or polarisation levels in ¹³C-alanine can be obtained by using a specific base for the preparation of the carboxylate salt of ¹³C-alanine.

The carboxylate salt of ¹³C-alanine may either be prepared and isolated or prepared in situ without isolating the obtained carboxylate salt of ¹³C-alanine. The advantage of isolating the salt before the DNP polarisation is that the isolated salt can be characterized and it can be determined how much of the carboxylate salt of ¹³C-alanine was actually converted into the carboxylate salt. Further, if other solvents are used in the DNP process than for the preparation of the carboxylate salt, it is preferred to isolate the carboxylate salt as well.

The DNP agent plays a decisive role in the DNP process as its choice has a major impact on the level of polarisation that can be achieved in ¹³C-alanine. A variety of DNP agents—in WO-A-99/35508 denoted “OMRI contrast agents”—is known like transition metals such as chromium (V) ions, magnetic particles or organic free radicals such as nitroxide radicals or trityl radicals. The use of oxygen-based, sulphur-based or carbon-based stable trityl radicals as described in WO-A-99/35508, WO-A-88/10419, WO-A-90/00904, WO-A-91/12024, WO-A-93/02711 or WO-A-96/39367 has resulted in high levels of polarisation in a variety of different chemical entities.

In a preferred embodiment, the hyperpolarised ¹³C-alanine used in the imaging medium of the invention is obtained by DNP and the DNP agent used is a trityl radical. As briefly mentioned above, the large electron spin polarisation of the DNP agent, i.e. trityl radical is converted to nuclear spin polarisation of ¹³C nuclei in ¹³C-alanine via microwave irradiation close to the electron Larmor frequency. The microwaves stimulate communication between electron and nuclear spin systems via e-e and e-n transitions. For effective DNP, i.e. to achieve a high level of polarisation in ¹³C-alanine, the trityl radical has to be stable and soluble in a solution of ¹³C-alanine to achieve said intimate contact between ¹³C-alanine and the trityl radical which is necessary for the aforementioned communication between electron and nuclear spin systems.

In a preferred embodiment, the trityl radical is a radical of the formula (1)

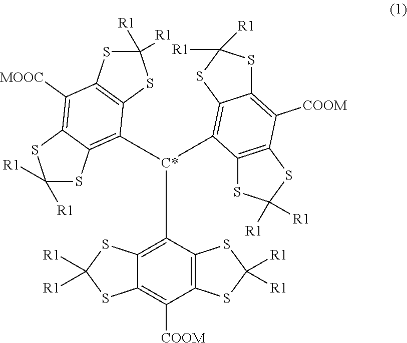

wherein
M represents hydrogen or one equivalent of a cation; and
R1 which is the same or different represents a straight chain or branched C₁-C₆-alkyl group optionally substituted by one or more hydroxyl groups or a group —(CH₂)ₙ—X—R2,
   wherein n is 1, 2 or 3;
   X is O or S; and
   R2 is a straight chain or branched C₁-C₄-alkyl group, optionally substituted by one or more hydroxyl groups.

In a preferred embodiment, M represents hydrogen or one equivalent of a physiologically tolerable cation. The term “physiologically tolerable cation” denotes a cation that is tolerated by the human or non-human animal living body. Preferably, M represents hydrogen or an alkali cation, an ammonium ion or an organic amine ion, for instance meglumine. Most preferably, M represents hydrogen or sodium.

In a preferred embodiment, R1 is the same, more preferably a straight chain or branched C₁-C₄-alkyl group, most preferably methyl, ethyl or isopropyl; or R1 is preferably the same, more preferably a straight chain or branched C₁-C₄-alkyl group which is substituted by one hydroxyl group, most preferably —CH₂—CH₂—OH; or R1 is preferably the same and represents —CH₂—OC₂H₄OH.

The aforementioned trityl radicals of formula (1) may be synthesized as described in detail in WO-A-88/10419, WO-A-90/00904, WO-A-91/12024, WO-A-93/02711, WO-A-96/39367, WO-A-97/09633, WO-A-98/39277 and WO-A-2006/011811.

Generally, for the DNP process, a liquid composition is prepared which comprises the starting material and the DNP agent. If the starting material or DNP agent is not a liquid, a solvent needs to be added to this composition. In the following the liquid composition for DNP is denoted “a composition for DNP”. To obtain hyperpolarised ¹³C-alanine by the DNP method, a composition for DNP is prepared which comprises the starting material, i.e. ¹³C-alanine or a salt thereof (in the following ¹³C-alanine or a salt thereof are denoted a sample) and the DNP agent, preferably a trityl radical, more preferably a trityl radical of formula (I). A solvent or a solvent mixture needs to be used to promote dissolution of the DNP agent and the sample. If the hyperpolarised ¹³C-alanine is intended to be used as imaging agent in an imaging medium for in vivo ¹³C-MR detection, it is preferred to keep the amount of solvent to a minimum. To be used in an in vivo imaging medium, the hyperpolarised ¹³C-alanine needs usually to be administered in relatively high concentrations, i.e. a highly concentrated sample is preferably used in the DNP process and hence the amount of solvent is preferably kept to a minimum. In this context, it is also important to mention that the mass of the composition containing the sample, i.e. DNP agent, sample and if necessary solvent, is kept as small as possible. A high mass will have a negative impact on the efficiency of the dissolution process, if dissolution is used to convert the solid composition containing the hyperpolarised sample after the DNP process into the liquid state, e.g. for using it in an imaging medium for ¹³C-MR detection. This is due to the fact that for a given volume of dissolution medium in the dissolution process, the mass of the composition to dissolution medium ratio decreases, when the mass of the composition increases. Further, using certain solvents may require their removal before the hyperpolarised ¹³C-alanine used in the imaging medium of the invention is administered to a human or non-human animal being since they might not be physiologically tolerable.

If the starting material used to obtain hyperpolarised ¹³C-alanine is an ammonium salt of ¹³C-alanine, e.g. the preferred ¹³C-alanininium chloride, said salt may be a commercially available salt which is dissolved in a suitable solvent, preferably water or a glass former like glycerol or glycol, or a mixture of water and a glass former. Alternatively, the ammonium salt it is preferably prepared and isolated before being used to prepare the composition for DNP. As an example, ¹³C₁-alaninium chloride may be prepared by adding hydrochloric acid to ¹³C₁-alanine, optionally in the presence of a solvent, for instance ethanol. The obtained ¹³C₁-alaninium chloride can for example be isolated by ether precipitation and dried. The obtained ¹³C₁-alaninium chloride is then dissolved in a suitable solvent, preferably water or a glass former like glycerol or glycol, or a mixture of water and a glass former. The DNP agent, preferably a trityl radical and more preferably a trityl radical of formula (I) may either be added to the dissolved ¹³C₁-alaninium chloride as a solid or in solution. Alternatively, the DNP agent is dissolved in a suitable solvent preferably water or a glass former like glycerol or glycol, or a mixture of water and a glass former and the solid ¹³C₁-alaninium chloride is added to the dissolved DNP agent. Intimate mixing of the compounds can be promoted by several means known in the art, such as stirring, vortexing or sonication and/or gentle heating.

If the starting material used to obtain hyperpolarised ¹³C-alanine is a carboxylate salt of ¹³C-alanine, e.g. the preferred sodium ¹³C-2-aminopropanoate, said salt may be a commercially available salt which is dissolved in a suitable solvent, preferably water or a glass former like glycerol or glycol, or a mixture of water and a glass former. Alternatively, it is preferably prepared in situ and used to prepare the composition for DNP without isolating it. As an example sodium ¹³C₁-2-aminopropanoate may be prepared by adding an aqueous solution of NaOH to ¹³C₁-alanine, optionally in the presence of a solvent, for instance water. To the obtained sodium ¹³C₁-2-aminopropanoate is then added the DNP agent, preferably a trityl radical and more preferably a trityl radical of formula (1), as a solid. Alternatively, the DNP agent is dissolved in a suitable solvent preferably water or a glass former like glycerol or glycol, or a mixture of water and a glass former and the dissolved DNP agent is then added to the obtained sodium ¹³C₁-2-aminopropanoate. Intimate mixing of the compounds can be promoted by several means known in the art, such as stirring, vortexing or sonication and/or gentle heating.

The composition of DNP may further comprise a paramagnetic metal ion. It has been found that the presence of paramagnetic metal ions may result in increased polarisation levels in the compound to be polarised by DNP as described in detail in WO-A2-2007/064226 which is incorporated herein by reference.

The term “paramagnetic metal ion” denotes paramagnetic metal ions in the form of their salts or in chelated form, i.e. paramagnetic chelates. The latter are chemical entities comprising a chelator and a paramagnetic metal ion, wherein said paramagnetic metal ion and said chelator form a complex, i.e. a paramagnetic chelate.

In a preferred embodiment, the paramagnetic metal ion is a salt or paramagnetic chelate comprising Gd³⁺, preferably a paramagnetic chelate comprising Gd³⁺. In a more preferred embodiment, said paramagnetic metal ion is soluble and stable in the solution of step a).

As with the DNP agent described before, the sample must be in intimate contact with the paramagnetic metal ion as well. A composition for DNP comprising the sample, a DNP agent and a paramagnetic metal ion may be obtained in several ways.

In a first embodiment the sample is dissolved in a suitable solvent to obtain a solution, alternatively the sample is generated in situ in a suitable solvent as described above. To these solutions of the sample the DNP agent is added and dissolved. The DNP agent, preferably a trityl radical, might be added as a solid or in solution, e.g. dissolved in a suitable solvent, preferably water or a glass former like glycerol or glycol, or a mixture of water and a glass former. In a subsequent step, the paramagnetic metal ion is added. The paramagnetic metal ion might be added as a solid or in solution, e.g. dissolved in a suitable solvent, preferably water or a glass former like glycerol or glycol, or a mixture of water and a glass former. In another embodiment, the DNP agent and the paramagnetic metal ion are dissolved in a suitable solvent and to this solution is added the sample, either as a solid or dissolved in a suitable solvent. In yet another embodiment, the DNP agent (or the paramagnetic metal ion) is dissolved in a suitable solvent and added to the optionally dissolved sample. In a subsequent step the paramagnetic metal ion (or the DNP agent) is added to this solution, either as a solid or in solution. Preferably, the amount of solvent to dissolve the paramagnetic metal ion (or the DNP agent) is kept to a minimum. Again intimate mixing of the compounds can be promoted by several means known in the art, such as stirring, vortexing or sonication and/or gentle heating.

If a trityl radical is used as DNP agent, a suitable concentration of such a trityl radical is 1 to 25 mM, preferably 2 to 20 mM, more preferably 10 to 15 mM in the composition used for DNP. If a paramagnetic metal ion is added to the composition, a suitable concentration of such a paramagnetic metal ion is 0.1 to 6 mM (metal ion) in the composition, and a concentration of 0.3 to 4 mM is preferred.

After having prepared the composition for DNP, said composition is frozen by methods known in the art, e.g. by freezing it in a freezer, in liquid nitrogen or by simply adding it to a probe-retaining cup (sample cup) and placing the sample cup in the DNP polariser, where liquid helium will freeze the composition. In another embodiment, the composition is frozen as “beads” before it is added to the sample cup and inserted into the polariser. Such beads may be obtained by adding the composition drop wise to liquid nitrogen. A more efficient dissolution of such beads has been observed, which is especially relevant if larger amounts of sample are polarised, for instance when the hyperpolarised ¹³C-alanine is intended to be used in an in vivo MR imaging medium.

If a paramagnetic metal ion is present in the composition said composition may be degassed before freezing, e.g. by bubbling helium gas through the composition (e.g. for a time period of 2-15 min) but degassing can be effected by other known common methods.

The DNP technique is for instance described in WO-A-98/58272 and in WO-A-01/96895, both of which are included by reference herein. Generally, a moderate or high magnetic field and a very low temperature are used in the DNP process, e.g. by carrying out the DNP process in liquid helium and a magnetic field of about 1 T or above. Alternatively, a moderate magnetic field and any temperature at which sufficient polarisation enhancement is achieved may be employed. In the context of this invention, the DNP process is carried out in liquid helium and a magnetic field of about 1 T or above. Suitable polarisation units are for instance described in WO-A-02/37132. In a preferred embodiment, the polarisation unit comprises a cryostat and polarising means, e.g. a microwave chamber connected by a wave guide to a microwave source in a central bore surrounded by magnetic field producing means such as a superconducting magnet. The bore extends vertically down to at least the level of a region P near the superconducting magnet where the magnetic field strength is sufficiently high, e.g. between 1 and 25 T, for polarisation of the NMR active sample nuclei to take place. The bore for the probe (i.e. the frozen composition to be polarised) is preferably sealable and can be evacuated to low pressures, e.g. pressures in the order of 1 mbar or less. A probe introducing means such as a removable transporting tube can be contained inside the bore and this tube can be inserted from the top of the bore down to a position inside the microwave chamber in region P. Region P is cooled by liquid helium to a temperature low enough to for polarisation to take place, preferably temperatures of the order of 0.1 to 100 K, more preferably 0.5 to 10 K, most preferably 1 to 5 K. The probe introducing means is preferably sealable at its upper end in any suitable way to retain the partial vacuum in the bore. A probe-retaining container, such as a probe-retaining cup or sample cup, can be removably fitted inside the lower end of the probe introducing means. The probe-retaining container is preferably made of a light-weight material with a low specific heat capacity and good cryogenic properties such, e.g. KelF (polychlorotrifluoro-ethylene) or PEEK (polyetheretherketone) and it may be designed in such a way that it can hold more than one probe.

The probe is inserted into the probe-retaining container, submerged in the liquid helium and irradiated with microwaves, preferably at a frequency of about 94 GHz at 200 mW. The level of polarisation may for instance be monitored by solid state ¹³C-NMR measurements of the ¹³C-nuclei in the frozen composition comprising the hyperpolarised sample. The solid state ¹³C-NMR measurement preferably consists of a simple pulse-acquire NMR sequence using a low flip angle. The signal intensity of the hyperpolarised sample in the ¹³C-NMR spectrum is compared with signal intensity of the sample in a ¹³C-NMR spectrum acquired before the DNP polarisation process. The level of polarisation is then calculated from the ratio of the signal intensities of before and after polarisation.

For use in an imaging medium, the frozen composition containing the hyperpolarised sample needs to be transferred from the solid state to the liquid state, i.e. liquefied after the dynamic nuclear polarisation.

Liquefaction can be achieved by dissolution in an appropriate solvent or solvent mixture (dissolution medium) or by melting the solid frozen composition. Dissolution is preferred and the dissolution process and suitable devices therefore are described in detail in WO-A-02/37132. The melting process and suitable devices for the melting are for instance described in WO-A-02/36005. Briefly, a dissolution unit/melting unit is used which is either physically separated from the polariser or is a part of an apparatus that contains the polariser and the dissolution unit/melting unit. In a preferred embodiment, dissolution/melting is carried out at an elevated magnetic field, e.g. inside the polariser, to improve the relaxation and retain a maximum of the hyperpolarisation. Field nodes should be avoided and low field may lead to enhanced relaxation despite the above measures.

If the sample used in the composition for DNP is an ammonium salt of ¹³C-alanine, said salt needs to be converted to the free ¹³C-alanine by reaction (neutralization) with a base. Said neutralization may be carried out simultaneously or subsequently to the liquefaction. Thus, in one embodiment the liquefaction is carried out by melting or dissolution of the frozen composition and conversion is carried out after the frozen composition was dissolved/melted. In another embodiment, liquefaction and conversion are carried out simultaneously, e.g. by dissolving the frozen composition in a dissolution medium which is or contains a compound that is capable of converting the hyperpolarised ammonium salt of ¹³C-alanine to ¹³C-alanine. Neutralization is generally carried out with a base. In principal any base that is a stronger base than the amino group in ¹³C-alanine can be used for neutralization. Preferred bases are inorganic bases, more preferred aqueous solutions of alkali metal or earth alkali metal hydroxides, hydrogen carbonates or carbonates, like aqueous solutions of NaOH, Na₂CO₃, NaHCO₃, KOH, CsOH, Ca(OH)₂ or Sr(OH)₂. The most preferred base is NaOH since it is cheap and readily available. Further, sodium cations are very well tolerated by the human or non-human animal body and thus sodium bases, and more preferably NaOH, are preferably used for neutralization if the hyperpolarised ¹³C-alanine is used in an in vivo MR imaging medium.

If the sample used in the composition for DNP is a carboxylate salt of ¹³C-alanine, said salt needs to be converted to the free ¹³C-alanine by reaction (neutralization) with an acid. We have observed high relaxation rates and hence loss of polarisation in hyperpolarised ¹³C-alanine in solutions with a pH above 7, i.e. basic solutions. Thus, if a carboxylate salt of ¹³C-alanine was used in the composition for DNP, the liquefaction and neutralization of the solid composition comprising the hyperpolarised carboxylate salt of ¹³C-alanine needs to be carried out carefully in order to avoid loss of polarisation. Neutralization may be carried out simultaneously or subsequently to the liquefaction, for the latter it must be taken care that neutralization is carried out quickly and directly after liquefaction. Thus, in one embodiment the liquefaction is carried out by melting or dissolution of the frozen composition and neutralization with an acid is quickly carried out after the frozen composition was dissolved/melted. In another embodiment, liquefaction and neutralization are carried out simultaneously, e.g. by dissolving the frozen composition in a dissolution medium which is or contains an acid that is capable of converting the hyperpolarised carboxylate salt of ¹³C-alanine to ¹³C-alanine. In yet another embodiment the acid is added to the probe-retaining cup which contains the frozen composition in the dynamic nuclear polarisation process. This can be done by freezing the composition for DNP in the probe-retaining cup, adding the acid on top of the frozen composition and freezing the acid. Alternatively, the acid may be frozen in the probe-retaining cup and the composition for DNP is added on top of the frozen acid and then frozen. This procedure results in close proximity of the acid needed for the neutralization and the carboxylate salt of ¹³C-alanine and when liquefying the frozen composition, immediate neutralization is taking place. In principal any acid that has a lower pKa than the carboxyl group in ¹³C-alanine can be used for neutralization. Preferred acids are strong acids, even more preferred strong mineral acids like hydrochloric acid (HCl), hydrobromic acid (HBr), hydroiodic acid (HI) or sulphuric acid (H₂SO₄). The most preferred acid is HCl since it is cheap and readily available. Further, chloride anions are very well tolerated by the human or non-human animal body and thus hydrochloric acid is preferably used for neutralization if the hyperpolarised ¹³C-alanine is used in an in vivo MR imaging medium.

As stated above, liquefaction is preferably carried out by dissolution using a dissolution medium that is or comprises a solvent or solvent mixture, preferably an aqueous carrier. In a preferred embodiment, a physiologically tolerable and pharmaceutically accepted aqueous carrier like water or saline is used. In a most preferred embodiment, the dissolution medium is or comprises a buffer solution, especially if the hyperpolarised ¹³C-alanine is used in an imaging medium for in vivo MR detection. For in vitro MR-detection, also non aqueous solvents or solvent mixtures may be used as or in the dissolution medium, for instance DMSO or methanol or mixtures comprising an aqueous carrier and a non aqueous solvent, for instance mixtures of DMSO and water or methanol and water. In another preferred embodiment, the dissolution medium may further comprise one or more compounds which are able to bind or complex free paramagnetic metal ions, e.g. chelating agents like DTPA or EDTA.

In a preferred embodiment, liquefaction is preferably carried out by dissolution with a dissolution medium, preferably a buffer solution that comprises a base or acid suitable for neutralization of carboxylate salts or ammonium salts of ¹³C-alanine, i.e. converting them to free ¹³C-alanine. If an ammonium salt of ¹³C-alanine has been used in the composition for DNP and preferably if the hyperpolarised ¹³C-alanine is intended to be used in an in vivo MR imaging medium, it is preferred to carry out dissolution by using a dissolution medium comprising a buffer solution with a pH of from about 6.8 to 7 and a base. Suitable buffer solutions are for instance phosphate buffer (KH₂PO₄/Na₂HPO₄), ACES, PIPES, imidazole/HCl, BES, MOPS, HEPES, TES, TRIS, BIS-TRIS, HEPPS or TRICIN. If a carboxylate salt of ¹³C-alanine has been used in the composition for DNP, and preferably if the hyperpolarised ¹³C-alanine is intended to be used in an in vivo MR imaging medium, it is preferred to carry dissolution by using a dissolution medium comprising a buffer solution with a pH slightly lower than physiological pH, i.e. a pH of from about 6.8 to 7.2, and an acid. Suitable buffer solutions are for instance phosphate buffer (KH₂PO₄/Na₂HPO₄), ACES, PIPES, imidazole/HCl, BES, MOPS, HEPES, TES, TRIS, BIS-TRIS, HEPPS or TRICIN.

Subsequent to liquefaction, the DNP agent, preferably a trityl radical, and the optional paramagnetic metal ion may be removed from the liquid containing the hyperpolarised sample or the hyperpolarised ¹³C-alanine. Removal of these compounds is preferred if the hyperpolarised ¹³C-alanine is intended for use in an imaging medium for in vivo MR detection. It is preferred to first convert the hyperpolarised sample to the free ¹³C-alanine and remove the DNP agent and the optional paramagnetic metal ion after said conversion has taken place.

Methods which are useful to remove the trityl radical and the paramagnetic metal ion are known in the art and described in detail in WO-A2-2007/064226 and WO-A1-2006/011809.

In a preferred embodiment the hyperpolarised ¹³C-alanine of the imaging medium according to the invention is obtained by dynamic nuclear polarisation of a composition for DNP that comprises a salt of ¹³C-alanine, preferably an ammonium or carboxylate salt of ¹³C-alanine and more preferred ¹³C-alaninium chloride or sodium ¹³C-2-aminopropanoate, a trityl radical of formula (I) and optionally a paramagnetic chelate comprising Gd³⁺.

The imaging medium according to the invention may be used as imaging medium for in vitro ¹³C-MR detection, e.g. ¹³C-MR detection of cell cultures, samples, ex vivo tissue or isolated organs derived from the human or non-human animal body. For this purpose, the imaging medium is provided as a composition that is suitable for being added to, for instance, cell cultures, samples like urine, blood or saliva, ex vivo tissues like biopsy tissues or isolated organs. Such an imaging medium preferably comprises in addition to the imaging agent, i.e. hyperpolarised ¹³C-alanine, a solvent which is compatible with and used for in vitro cell or tissue assays, for instance DMSO or methanol or solvent mixtures comprising an aqueous carrier and a non aqueous solvent, for instance mixtures of DMSO and water or a buffer solution or methanol and water or a buffer solution. As it is apparent for the skilled person, pharmaceutically acceptable carriers, excipients and formulation aids may be present in such an imaging medium but are not required for such a purpose.

Further, the imaging medium according to the invention may be used as imaging medium for in vivo ¹³C-MR detection, i.e. ¹³C-MR detection carried out on living human or non-human animal beings. For this purpose, the imaging medium needs to be suitable for administration to a living human or non-human animal body. Hence such an imaging medium preferably comprises in addition to the imaging agent, i.e. hyperpolarised ¹³C-alanine, an aqueous carrier, preferably a physiologically tolerable and pharmaceutically accepted aqueous carrier like water, a buffer solution or saline. Such an imaging medium may further comprise conventional pharmaceutical or veterinary carriers or excipients, e.g. formulation aids such as stabilizers, osmolality adjusting agents, solubilising agents and the like which are conventional for diagnostic compositions in human or veterinary medicine.

If the imaging medium of the invention is used for in vivo ¹³C-MR detection, i.e. in a living human or non-human animal body, said imaging medium is preferably administered to the human or non-human animal body parenterally, preferably intravenously. Generally, the body under examination is positioned in an MR magnet. Dedicated ¹³C-MR RF-coils are positioned to cover the area of interest. Exact dosage and concentration of the imaging medium will depend upon a range of factors such as toxicity and the administration route. Generally, the imaging medium is administered in a concentration of up to 1 mmol ¹³C-alanine per kg bodyweight, preferably 0.01 to 0.5 mmol/kg, more preferably 0.1 to 0.3 mmol/kg. At less than 400 s after the administration, preferably less than 120 s, more preferably less than 60 s after the administration, especially preferably 20 to 50 s an MR imaging sequence is applied that encodes the volume of interest in a combined frequency and spatial selective way. The exact time of applying an MR sequence is highly dependent on the volume of interest.

The imaging medium according to the invention is preferably used in a method of ¹³C-MR detection and such a method is another aspect of the invention.

Thus, in a second aspect the invention provides a method of ¹³C-MR detection using an imaging medium comprising hyperpolarised ¹³C-alanine.

In a preferred first embodiment, the invention provides a method of ¹³C-MR detection using an imaging medium comprising hyperpolarised ¹³C-alanine wherein signals of ¹³C-lactate and optionally of ¹³C-alanine and/or ¹³C-pyruvate and/or ¹³C-bicarbonate are detected.

The term “signals of ¹³C-lactate and optionally ¹³C-alanine and/or ¹³C-pyruvate and/or ¹³C-bicarbonate are detected” means that in the method of the invention, only the signal of ¹³C-lactate is detected or the signals of ¹³C-lactate and ¹³C-alanine, or ¹³C-lactate and ¹³C-pyruvate or ¹³C-lactate and ¹³C-bicarbonate are detected or the signals of ¹³C-lactate and ¹³C-alanine and ¹³C-pyruvate or ¹³C-lactate and ¹³C-alanine and ¹³C-bicarbonate or ¹³C-lactate and ¹³C-pyruvate and ¹³C-bicarbonate are detected or the signals of ¹³C-lactate and ¹³C-alanine and ¹³C-pyruvate and ¹³C-bicarbonate are detected.

The term “¹³C-lactate” denotes a salt of lactic acid that is isotopically enriched with ¹³C, i.e. in which the amount of ¹³C isotope is greater than its natural abundance. Unless otherwise specified, the term “¹³C-lactate” denotes a compound which is ¹³C-enriched at the C1- and/or C2- and/or C3-position. The position of the isotopic enrichment in ¹³C-lactate is of course dependent on the position of the isotopic enrichment in its parent compound ¹³C-alanine. Thus, if for example hyperpolarised ¹³C₁-alanine was used in the imaging medium used in the method of the invention, the signal of ¹³C₁-lactate is detected.

The term “¹³C-pyruvate” denotes a salt of pyruvic acid that is isotopically enriched with ¹³C, i.e. in which the amount of ¹³C isotope is greater than its natural abundance. Unless otherwise specified, the term “¹³C-pyruvate” denotes a compound which is ¹³C-enriched at the C1- and/or C2- and/or C3-position The position of the isotopic enrichment in ¹³C-pyruvate is of course dependent on the position of the isotopic enrichment in its parent compound ¹³C-alanine. Thus, if for example hyperpolarised ¹³C₁-alanine was used in the imaging medium used in the method of the invention, the signal of ¹³C₁-pyruvate is detected.

The term “¹³C-bicarbonate” denotes a HCO₃⁻ that is isotopically enriched with ¹³C, i.e. in which the amount of ¹³C isotope is greater than its natural abundance. ¹³C-bicarbonate can only be detected if the parent compound ¹³C-alanine was isotopically enriched at the C1-position.

The metabolic conversion of alanine to pyruvate and lactate is shown in Scheme 1 for ¹³C₁-alanine; * denotes the ¹³C-label: ¹³C-pyruvate is formed by transamination of ¹³C-alanine with α-ketoglutarate, a reaction which is catalysed by alanine transaminase (ALT, EC 2.6.1.2). Further, ¹³C-pyruvate is formed by transamination of ¹³C-alanine with glyoxylate, a reaction which is catalysed by alanine-glyoxylate transaminase (AGT, EC 2.6.1.44). ¹³C-pyruvate is converted to ¹³C-lactate by lactate dehydrogenase (LDH, EC 1.1.1.27) and to ¹³C-bicarbonate by the pyruvate dehydrogenase complex (PDC).

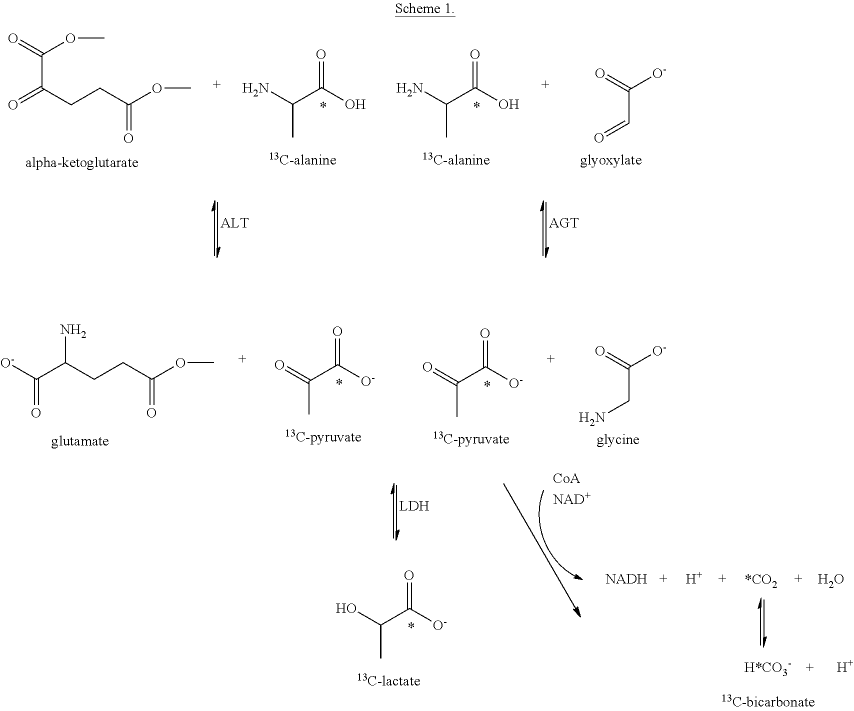

The term “signal” in the context of the invention refers to the MR signal amplitude or integral or peak area to noise of peaks in a ¹³C-MR spectrum which represent ¹³C-lactate and optionally ¹³C-alanine and/or ¹³C-pyruvate and/or ¹³C-bicarbonate. In a preferred embodiment, the signal is the peak area.

In a preferred embodiment of the method of the invention, the above-mentioned signals of ¹³C-lactate and optionally ¹³C-alanine and/or ¹³C-pyruvate and/or ¹³C-bicarbonate are used to generate a metabolic profile of a living human or non-human animal being. Said metabolic profile may be derived from the whole body, e.g. obtained by whole body in vivo ¹³C-MR detection. Alternatively, said metabolic profile is generated from a region or volume of interest, i.e. a certain tissue, organ or part of said human or non-human animal body.

In another preferred embodiment of the method of the invention, the above-mentioned signals of ¹³C-lactate and optionally ¹³C-alanine and/or ¹³C-pyruvate and/or ¹³C-bicarbonate are used to generate a metabolic profile of cells in a cell culture, of samples like urine, blood or saliva, of ex vivo tissue like biopsy tissue or of an isolated organ derived from a human or non-human animal being. Said metabolic profile is then generated by in vitro ¹³C-MR detection.

Thus, in a preferred first embodiment, the invention provides a method of ¹³C-MR detection using an imaging medium comprising hyperpolarised ¹³C-alanine wherein signals of ¹³C-lactate and optionally of ¹³C-alanine and/or ¹³C-pyruvate and/or ¹³C-bicarbonate are detected and wherein said signals are used to generate a metabolic profile.

In a more preferred first embodiment, the signals of ¹³C-lactate and ¹³C-alanine are used to generate said metabolic profile. In another more preferred embodiment, the signals of ¹³C-lactate and ¹³C-alanine and ¹³C-pyruvate and/or ¹³C-bicarbonate are used to generate said metabolic profile.

In one embodiment, the spectral signal intensities of ¹³C-lactate and optionally of ¹³C-alanine and/or ¹³C-pyruvate and/or ¹³C-bicarbonate are used to generate the metabolic profile. In another embodiment, the spectral signal integrals of ¹³C-lactate and optionally of ¹³C-alanine and/or ¹³C-pyruvate and/or ¹³C-bicarbonate are used to generate the metabolic profile. In another embodiment, signal intensities from separate images of ¹³C-lactate and optionally of ¹³C-alanine and/or ¹³C-pyruvate and/or ¹³C-bicarbonate are used to generate the metabolic profile. In yet another embodiment, the signal intensities of ¹³C-lactate and optionally of ¹³C-alanine and/or ¹³C-pyruvate and/or ¹³C-bicarbonate are obtained at two or more time points to calculate the rate of change of ¹³C-lactate and optionally the rate of change of ¹³C-alanine and/or ¹³C-pyruvate and/or ¹³C-bicarbonate.

In another embodiment the metabolic profile includes or is generated using processed signal data of ¹³C-lactate and optionally of ¹³C-alanine and/or ¹³C-pyruvate and/or ¹³C-bicarbonate, e.g. ratios of signals, corrected signals, or dynamic or metabolic rate constant information deduced from the signal pattern of multiple MR detections, i.e. spectra or images.

Hence, in a preferred embodiment a corrected ¹³C-lactate signal, i.e. ¹³C-lactate to ¹³C-alanine signal and/or ¹³C-lactate to ¹³C-pyruvate and/or ¹³C-lactate to ¹³C-bicarbonate signal is included into or used to generate the metabolic profile. In a further preferred embodiment, a corrected ¹³C-lactate to total ¹³C-carbon signal is included into or used to generate the metabolic profile with total ¹³C-carbon signal being the sum of the signals of ¹³C-lactate and ¹³C-alanine and/or ¹³C-pyruvate and/or ¹³C-bicarbonate. In a more preferred embodiment, the ratio of ¹³C-lactate to ¹³C-alanine and/or ¹³C-pyruvate and/or ¹³C-bicarbonate is included into or used to generate the metabolic profile.

The metabolic profile generated in the preferred embodiment of the method according to the invention provides information about the metabolic activity of the body, part of the body, cells, tissue, body sample etc under examination and said information may be used in a subsequent step for, e.g. identifying diseases.

Such a disease may be a tumour since tumour tissue is usually characterized by a higher metabolic activity than healthy tissue. Such a higher metabolic activity would be apparent from comparing the metabolic profile of a tumour or of an ex vivo sample of a tumour with the metabolic profile of healthy tissue (e.g. surrounding tissue or healthy ex vivo tissue) and may manifest itself in the metabolic profile by high signals of the ¹³C-lactate or high corrected ¹³C-lactate signal or ratio of ¹³C-alanine to ¹³C-lactate or high metabolic rate of ¹³C-lactate build-up.

The term “high” is a relative term and it has to be understood that the “high signal, ratio, metabolic rate” etc. which is seen in a metabolic profile of a diseased tissue as described above is increased compared to the signal, ratio, metabolic rate etc. which is seen in a metabolic profile of a healthy tissue.

Another disease may be ischemia in the heart since ischemic myocardial tissue usually is characterized by a lower metabolic activity than healthy myocardial tissue. Again such a lower metabolic activity would be apparent from comparing the metabolic profile of ischemic myocardial tissue with the metabolic profile of healthy myocardial tissue in a way as described in the previous paragraph.

Yet another disease may be liver related diseases, such as diabetes, liver fibrosis or cirrhosis. In these diseases serum alanine transaminase is a sensitive predictor of mortality. Metabolic profiles can be compared between healthy liver and diseased liver just as described above.

Anatomical and/or—where suitable—perfusion information may be included in the method of the invention for identification of diseases. Anatomical information may for instance be obtained by acquiring a proton or ¹³C-MR image with or without employing a suitable contrast agent before or after the method of the invention.

In another preferred embodiment, the imaging medium comprising hyperpolarised ¹³C-alanine is administered repeatedly, thus allowing dynamic studies. This is a further advantage of the method according to the invention compared to other MR detection methods using conventional MR contrast agents which—in higher doses—may show toxic effects. Alanine is present in the human body and hyperpolarised ¹³C-alanine was well tolerated in the animal models we have used and described in the Examples part of this application. Hence it is expected that hyperpolarised ¹³C-alanine will be well tolerated in patients as well and thus administration of repeated doses of this compound should be possible.

As stated above, the metabolic profile provides information about the metabolic activity of the body, part of the body, cells, tissue, body sample etc. under examination and said information may be used in a subsequent step for, e.g. identifying diseases. However, a physician may also use this information in a further step to choose the appropriate treatment for the patient under examination.

Thus, said information may be used to monitor treatment response, e.g. treatment success of the above mentioned diseases, and its sensitivity makes the method especially suitable for monitoring early treatment response, i.e. response to treatment shortly after its commencement.

In yet another embodiment, the method of the invention may be used to assess drug efficacy. In said embodiment, potential drugs for curing a certain disease like for instance anti-cancer drugs, may be tested at a very early stage in drug screening, for instance in vitro in a cell culture which is a relevant model for said certain disease or in diseased ex vivo tissue or a diseased isolated organ. Alternatively, potential drugs for curing a certain disease may be tested at an early stage in drug screening in vivo, for instance in an animal model which is relevant for said certain disease. By comparing the metabolic profile of said cell culture, ex vivo tissue, isolated or test animal before and after treatment with a potential drug, the efficacy of said drug and thus treatment response and success can be determined which of course provides valuable information in the screening of potential drugs.

Yet another aspect of the invention is a composition comprising ¹³C-alanine, a DNP agent and optionally a paramagnetic metal ion. Said composition can be used for obtaining hyperpolarised ¹³C-alanine by dynamic nuclear polarisation (DNP) which can be used as imaging agent (MR active agent) in the imaging medium according to the invention.

In preferred embodiment, the composition according to the invention comprises a salt of ¹³C-alanine, preferably an ammonium salt or carboxylate salt of ¹³C-alanine and more preferably ¹³C-alaninium chloride or sodium ¹³C-2-aminopropanoate. It is further preferred that the ¹³C-alanine or salt of ¹³C-alanine is a ¹³C₁-alanine or salt of ¹³C₁-alanine. In another preferred embodiment, the DNP agent in the composition according to the invention is a trityl radical, more preferably a trityl radical of formula (1) and most preferably a trityl radical of formula (1) and most preferably a trityl radical of formula (1) wherein M represents hydrogen or sodium and R1 is preferably the same, more preferably a straight chain or branched C₁-C₄-alkyl group, most preferably methyl, ethyl or isopropyl; or R1 is preferably the same, more preferably a straight chain or branched C₁-C₄-alkyl group which is substituted by one hydroxyl group, most preferably —CH₂—CH₂—OH; or R1 is preferably the same and represents —CH₂—OC₂H₄OH. In another preferred embodiment said composition comprises a paramagnetic metal ion, preferably a salt or paramagnetic chelate comprising Gd³⁺ and more a paramagnetic chelate comprising Gd³⁺. Suitably, said composition further comprises a solvent or solvents and/or a glass former. If the composition comprises a salt of ¹³C-alanine, it preferably also comprises a glass former like for instance glycerol. The aforementioned compositions can be used for obtaining hyperpolarised ¹³C-alanine by dynamic nuclear polarisation (DNP) with a high polarisation level. If the composition comprises a salt of ¹³C-alanine, the hyperpolarised salt of ¹³C-alanine can be converted into hyperpolarised ¹³C-alanine by neutralization with an acid or a base as described earlier in the application.

Yet another aspect of the invention is a composition comprising hyperpolarised ¹³C-alanine or a hyperpolarised salt of ¹³C-alanine, a DNP agent and optionally a paramagnetic metal ion, wherein said composition is obtained by dynamic nuclear polarisation of a composition as described in the previous paragraphs. Preferred embodiments of said composition comprising hyperpolarised ¹³C-alanine or a hyperpolarised salt of ¹³C-alanine, a DNP agent and optionally a paramagnetic metal ion are also described in the previous paragraph.

Yet another aspect of the invention is hyperpolarised ¹³C-alanine or a hyperpolarised salt of ¹³C-alanine. A preferred embodiment of the latter is a hyperpolarised ammonium salt or carboxylate salt of ¹³C-alanine and more preferably hyperpolarised ¹³C-alaninium chloride or sodium ¹³C-2-aminopropanoate. It is further preferred that the hyperpolarised ¹³C-alanine or salt of ¹³C-alanine is a hyperpolarised ¹³C₁-alanine or hyperpolarised salt of ¹³C₁-alanine The aforementioned compounds can be used as imaging agent (MR active agent) in the imaging medium according to the invention and said imaging medium can be used in the methods of ¹³C-MR detection according to the invention.

Yet another aspect of the invention is a method for producing hyperpolarised ¹³C-alanine, the method comprising preparing a composition comprising ¹³C-alanine or a salt of ¹³C-alanine, a DNP agent and optionally a paramagnetic metal ion and carrying out dynamic nuclear polarisation on said composition. If a salt of ¹³C-alanine has been when preparing the composition, the free ¹³C-alanine is obtained by neutralizing the composition after DNP. The preparation of said composition and how to carry out dynamic nuclear on said composition is described in detail earlier in the application.

### BRIEF DESCRIPTION OF THE DRAWINGS

FIG. 1 depicts signal intensities of ¹³C₁-alanine and ¹³C₁-lactate, over time detected from ¹³C-MR spectroscopy imaging of mice (whole body).

FIG. 2 depicts a stacked plot of 5 ¹³C-MR scans showing the signal intensities of ¹³C₁-alanine (177.0 ppm) and ¹³C₁-lactate (183.7 ppm).

FIG. 3 depicts signal intensities of ¹³C₁-alanine and ¹³C₁-lactate over time detected from ¹³C-MR spectroscopy imaging of mouse liver.

FIG. 4 depicts a combined ¹³C-MR spectrum of 15 separate ¹³C-MR scans showing the signal intensities of ¹³C₁-alanine (177.0 ppm), ¹³C₁-lactate (183.7 ppm), ¹³C₁-pyruvate (171.6 ppm) and ¹³C₁-bicarbonate (161.5 ppm).

FIG. 5 depicts signal intensities of ¹³C₁-alanine and ¹³C₁-lactate over time detected from ¹³C-MR spectroscopy imaging of mouse heart.

The invention is illustrated by the following non-limiting examples.

### EXAMPLES

### Example 1

### Preparation of ¹³C₁-Alanine

### Example 1a

### Preparation of an Ammonium Salt of ¹³C₁-Alanine (¹³C₁-Alaninium Chloride)

¹³C₁-alanine (100 mg, 1.1 mol, Cambridge Isotopes) was added to a 10 ml centrifugal tube, followed by addition of concentrated hydrochloric acid (145 μl, 12 M) and ethanol (1 ml, 95%). After dissolution of the ¹³C₁-alanine (sonication may be required) the resulting ¹³C₁-alaninium chloride was precipitated by the addition of diethyl ether (approx. 5 ml). The precipitation was collected by centrifugation and the supernatant was discarded. The precipitation was washed with diethyl ether and dried in vacuo. Recovered yield: 125 mg white powder (90%, as fine needles).

### Example 1b

### Preparation and DNP Polarisation of a Composition Comprising ¹³C₁-Alaninium Chloride, a DNP Agent and a Paramagnetic Metal Ion

32.5 mg (0.258 mmol) of the ¹³C₁-alaninium chloride obtained in Example 1a was added to 42 mg of a stock solution in a micro test tube. The stock solution had been prepared by dissolving the DNP agent (trityl radical) tris(8-carboxy-2,2,6,6-(tetra(hydroxyethyl)-benzo-[1,2-4,5′]-bis-(1,3)-dithiole-4-yl)-methyl sodium salt which had been synthesised according to Example 7 of WO-A1-98/39277 and the paramagnetic metal ion (Gd-chelate of 1,3,5-tris-(N-(DO3A-acetamido)-N-methyl-4-amino-2-methylphenyl)-[1,3,5]triazinane-2,4,6-trione) which had been synthesised according to Example 4 of WO-A-2007/064226 in glycerol in such a way that a glycerol solution being 26 mM in trityl radical and 0.52 mM in Gd-chelate had been obtained. The resulting composition was sonicated to dissolve the ¹³C₁-alanine hydrochloride and produce a clear solution. The solution (65 μl, 4 M in ¹³C₁-alaninium chloride, 17 mM in trityl radical and 0.9 mM in Gd³⁺) was transferred with a pipette into a sample cup which was quickly lowered into liquid nitrogen to freeze the solution and then inserted into a DNP polariser. The frozen solution was polarised under DNP conditions at 1.2 K in a 3.35 T magnetic field under irradiation with microwave (93.90 GHz). Polarisation was followed by solid state ¹³C-NMR and the solid state polarisation was determined to be 40%.

### Example 1c

### Liquefaction and Neutralization

After 150 minutes of dynamic nuclear polarisation, the obtained frozen polarised solution was dissolved in a dissolution medium containing 6 ml of a phosphate buffer (20 mM, pH 6.8, 100 mg/l EDTA), aqueous NaOH (27 μl 12 M solution, 1 eq) and 30 mg NaCl. The pH of the final liquid was 6.8.

Liquid state polarisation was determined by liquid state ¹³C-NMR at 400 MHz to be 35%.

### Example 2

### Preparation of Hyperpolarised ¹³C₁-Alanine

### Example 2b

### Preparation and DNP Polarisation of a Solution Comprising a Carboxylate Salt of ¹³C₁-alanine (Sodium ¹³C₁-2-Amino-Propanoate), a DNP Agent and a Paramagnetic Metal Ion

¹³C₁-alanine (21.6 mg, 0.24 mmol) was weighted into a micro test tube and dissolved in 20 μl aqueous NaOH (12 M). The mixture was sonicated and gently heated to produce a clear solution. To the solution was added 3.8 μl of an aqueous solution of tris(8-carboxy-2,2,6,6-(tetra(hydroxyethyl)-benzo-[1,2-4,5′]-bis-(1,3)-dithiole-4-yl)-methyl sodium salt (trityl radical; 143 mM) and 1.5 μl of an aqueous solution of the Gd-chelate of 1,3,5-tris-(N-(DO3A-acetamido)-N-methyl-4-amino-2-methylphenyl)-[1,3,5]triazinane-2,4,6-trione) (paramagnetic metal ion; 5 mM) The resulting composition was sonicated and gently heated to produce a clear solution. The solution (approx. 38 μl, 6 M in sodium ¹³C₁-2-amino-propanoate, 12.5 mM in trityl radical and 0.15 mM in Gd³⁺) was transferred with a pipette into a sample cup which was quickly lowered into liquid nitrogen to freeze the solution. The sample cup was removed from the liquid nitrogen, 22 μl aqueous HCl (12 M) were added to the sample cup. The sample cup was quickly lowered into liquid nitrogen again and then inserted into a DNP polariser. The frozen composition was polarised under DNP conditions at 1.2 K in a 3.35 T magnetic field under irradiation with microwave (93.90 GHz). Polarisation was followed by solid state ¹³C-NMR and the solid state polarisation was determined to be 18%.

### Example 2b

### Liquefaction and Neutralization

After 120 minutes of dynamic nuclear polarisation, the obtained frozen polarised solution was dissolved in a dissolution medium containing 6 ml BIS TRIS (40 mM, pH 6, 100 mg/l EDTA, 0.9% NaCl). The pH of the final solution containing the dissolved composition was 6.

Liquid state polarisation was determined by liquid state ¹³C-NMR at 400 MHz to be 16%.

### Example 3

### In Vitro ¹³C-MR Spectroscopy Using an Imaging Medium Comprising Hyperpolarised ¹³C₁-Alanine

An imaging medium was prepared as described in Example 1 and 100 μl of the imaging medium (3 mM ¹³C₁-alanine) was mixed into 10×10⁶ Hep-G2 cells (human hepatocellular carcinoma cells). A single ¹³C-MR spectrum was acquired after a total of 20 s incubation time with a 90 degree RF pulse. ¹³C₁-pyruvate and ¹³C₁-lactate were identified in the spectrum. The conversion of alanine was approximately 0.1% to both lactate and pyruvate.

### Example 4

### In Vivo ¹³C-MR Spectroscopy in Mice (Whole Body) Using an Imaging Medium Comprising Hyperpolarised ¹³C₁-Alanine

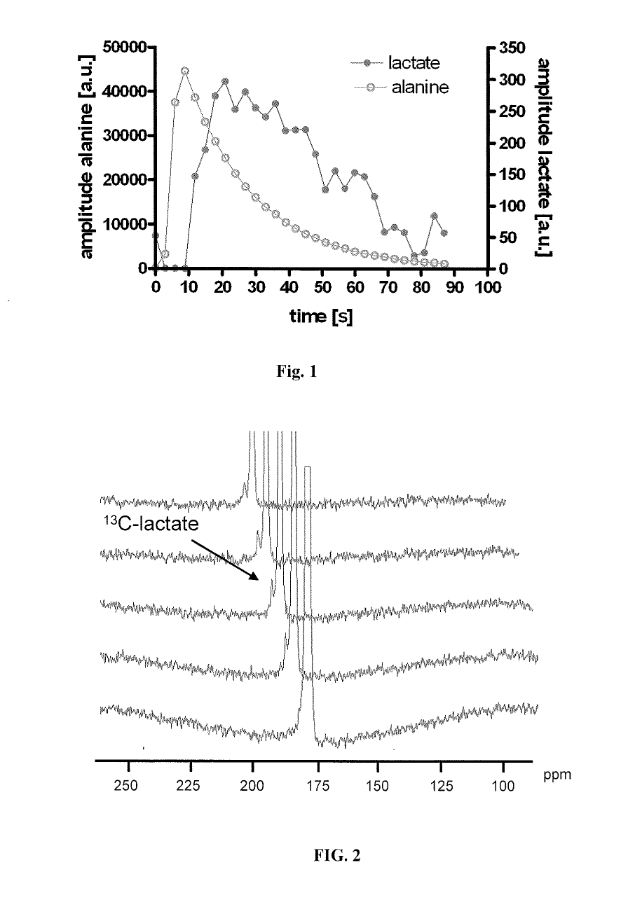

175 μl of an imaging medium which was prepared as described in Example 1 was injected into a C57B1/6 mouse over a time period of 6 s. The ¹³C₁-alanine concentration in said imaging medium was approximately 50 mM and 3 animals were used in the experiment. A rat size whole body coil (tuned for proton and carbon) was placed over the animal and ¹³C-MR spectroscopy was carried out in a 9.4 T magnet. A dynamic set of ¹³C-MR spectra (in total 5) was acquired every 5 s with a 15 degree RF pulse. Metabolism was seen to ¹³C₁-lactate (approximately 1% of the ¹³C₁-lactate signal), see FIG. 1. FIG. 2 shows a stacked plot of 5 acquired spectra. The following decay time was calculated from the MR spectra: ¹³C₁-lactate 33 s. No pyruvate signal could be detected, which is an indicator that the conversion of pyruvate to lactate is fast. It is thus favourable to detect the lactate signal which due to its slow decay provides a larger MR detection window.

### Example 5

### In Vivo ¹³C-MR Spectroscopy in Mice (Liver) Using an Imaging Medium Comprising Hyperpolarised ¹³C₁-Alanine

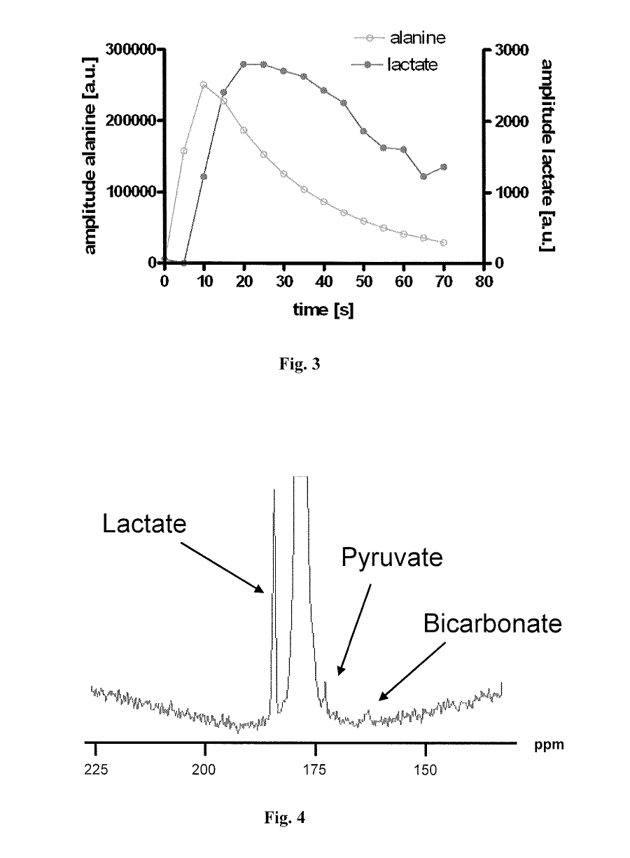

175 μl of an imaging medium which was prepared as described in Example 1 was injected into a C57B1/6 mouse over a time period of 6 s. The ¹³C₁-alanine concentration in said imaging medium was approximately 55 mM. A surface coil (tuned for proton and carbon) was positioned over the liver of the animal and ¹³C-MR spectroscopy was carried out in a 9.4 T magnet. A dynamic set of ¹³C-MR spectra (in total 15) was acquired every 5 s with a 30 degree RF pulse. This experiment confirmed that ¹³C₁-lactate is building up during the time course of the experiment, see FIG. 3. In this experiment (liver) also ¹³C₁-pyruvate and ¹³C₁-bicarbonate could be detected and FIG. 4 shows a combined spectrum of the 15 collected MR spectra.

### Example 6

### In Vivo ¹³C-MR Spectroscopy in Mice (Heart) Using an Imaging Medium Comprising Hyperpolarised ¹³C₁-Alanine

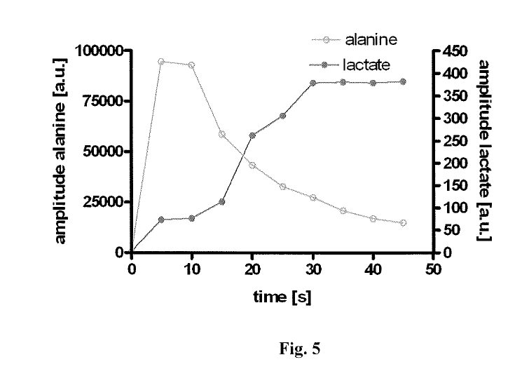

175 μl of an imaging medium which was prepared as described in Example 1 was injected into a C57B1/6 mouse over a time period of 6 s. The ¹³C₁-alanine concentration in said imaging medium was about 55 mM. A surface coil (tuned for proton and carbon) was positioned over the heart of the animal and ¹³C-MR spectroscopy was carried out in a 9.4 T magnet. A dynamic set of ¹³C-MR spectra (in total 10) was acquired every 5 s with a 30 degree RF pulse. In this experiment (heart) the build-up of ¹³C₁-lactate is noteworthy slow and the signal does not decay during the time course of the experiment, FIG. 5. The lactate seen in the experiment is expected to originate from pyruvate. The absence of pyruvate in the experiment suggests that pyruvate is instantaneously converted to lactate in the myocardium. Comparing Examples 5 and 6, a different metabolic profile is obtained for the liver and the heart showing that ¹³C-alanine is a tissue specific metabolic marker.

What is claimed is:

1. Imaging medium comprising hyperpolarised ¹³C-alanine.

2. The imaging medium according to claim 1 wherein said hyperpolarised ¹³C-alanine is hyperpolarised ¹³C₁-alanine.

3. The imaging medium according to claim 1 for use in in vivo or in vitro ¹³C-MR detection.

4. Method of ¹³C-MR detection comprising administering an imaging medium comprising hyperpolarised ¹³C-alanine; and detecting signals of ¹³C-lactate and optionally of ¹³C-alanine and/or ¹³C-pyruvate.

5. (canceled)

6. The method according to claim 4 wherein said signals are used to generate a metabolic profile.

7. The method according to claim 6 wherein said method is a method of in vivo ¹³C-MR detection and said metabolic profile is a metabolic profile of a living human or non-human animal being.

8. The method according to claim 6 wherein said method is a method of in vitro ¹³C-MR detection and said metabolic profile is a metabolic profile of cells in a cell culture, of samples, of ex vivo tissue or of an isolated organ.

9. Method according to claim 6 wherein the metabolic profile is used for identifying diseases.

10. Composition comprising ¹³C-alanine or a salt of ¹³C-alanine, a DNP agent and optionally a paramagnetic metal ion.

11. The composition according to claim 10 wherein said paramagnetic metal ion is present and is a paramagnetic chelate comprising Gd³⁺.

12. The composition according to claim 10 wherein said DNP agent is a trityl radical of formula (1) 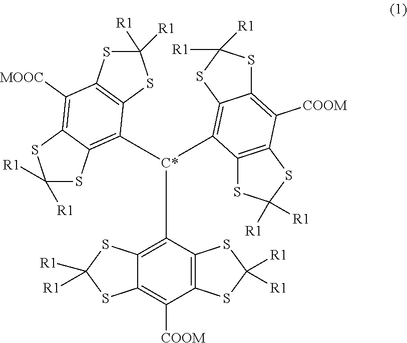
wherein
M represents hydrogen or one equivalent of a cation; and
R1 which is the same or different represents a straight chain or branched C₁-C₆-alkyl group optionally substituted by one or more hydroxyl groups or a group —(CH₂)ₙ—X—R2,
wherein n is 1, 2 or 3;
X is O or S; and
R2 is a straight chain or branched C₁-C₄-alkyl group, optionally substituted by one or more hydroxyl groups.

13. (canceled)

14. Composition comprising hyperpolarised ¹³C-alanine or a hyperpolarised salt of ¹³C-alanine, a DNP agent and optionally a paramagnetic metal ion, wherein said composition is obtained by dynamic nuclear polarisation of the composition of claim 10.

15. Hyperpolarised ¹³C-alanine or a hyperpolarised salt of ¹³C-alanine.
